(19) United States
(12) Patent Application Publication
(10) Pub. No.: US 2007/0032811 A1
(43) Pub. Date: Feb. 8, 2007

(54) Medical cutting and/or holding instrument

(76) Inventor: Martin Storz, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/509,298

(22) Filed: Aug. 24, 2006

Related U.S. Application Data
(63) Continuation of application No. PCT/EP05/01763, filed on Feb. 21, 2005.

(30) Foreign Application Priority Data
Feb. 25, 2004 (DE) 10 2004 009 200.1

(51) Int. Cl. A61B 17/32

(52) U.S. Cl. 606/174

(57) ABSTRACT

The invention relates to a medical cutting and/or holding instrument having a shaft, a handle consisting of two gripping members that is positioned on the proximal end of the shaft, and a push-pull rod mounted in a guide groove of the shaft to actuate a tool consisting of two jaw members that is positioned on the distal end of the shaft, such that for opening and closing the tool the push-pull rod can be coupled on the distal end with at least one rotatable jaw member of the tool and on the proximal end with a rotatable gripping member of the handle, and such that the push-pull rod and the rotatable gripping member can be dissolubly connected with one another by a coupling mechanism. To produce a medical cutting and/or holding instrument that can also be used for endoscopic purposes, it is proposed with the invention that the push-pull rod is coupled with the rotatable components both on the proximal and the distal ends with free play in a direction essentially perpendicular to the direction of motion of the push-pull rod.

### CROSS-REFERENCE TO RELATED APPLICATIONS

The present application is a continuation of pending International patent application PCT/EP2005/001763 filed on Feb. 21, 2005 which designates the United States and claims priority from German patent application 10 2004 009 200.1 filed on Feb. 25, 2004, the content of which is incorporated herein by reference.

### FIELD OF THE INVENTION

The invention relates to a medical cutting and/or holding instrument having a shaft, a handle consisting of two gripping members positioned on the proximal end of the shaft, and a push-pull rod mounted in a guide channel of the shaft for actuating a tool on the distal end of the shaft that consists of two jaw members, such that for opening and closing the tool the push-pull rod can be coupled on the distal end with at least one rotatable jaw member of the tool and on the proximal end with a rotatable gripping member of the handle, and such that the push-pull rod and the rotatable gripping member can be dissolubly connected to one another by a coupling mechanism.

### BACKGROUND OF THE INVENTION

Medical cutting and/or holding instruments are used in a range of applications both in open surgery and in endoscopic surgery. Because of the very narrow construction of endoscopic instruments, dictated by their use, since they are as a rule inserted through hollow trocar sleeves into the surgical area, so-called tubular shaft instruments have become established and proven in endoscopic surgery for the configuration of cutting and/or holding instruments. These tubular shaft instruments are distinguished in that the instrument shaft takes the form of a hollow tube and the push-pull rod provided to actuate the distal-end jaw member is positioned so that it can slide in the interior of the hollow shaft tube.

Because medical instruments, especially those coming in contact with blood, which are not designed as throwaway instruments, require thorough cleaning and sterilization, these hollow shaft instrument must be configured so that they can be disassembled in order to ensure, in particular, a reliable cleaning of the hollow tubular shaft. These structural features, which allow the medical instrument to be broken down into at least three parts—that is, the hollow tubular shaft, the push-pull rod, and the handle—make the production of this type of medical instrument more costly and make them difficult to assemble and disassemble.

Patent DE 94 21 125 U1 teaches a generic medical cutting and/or holding instrument in which the push-pull rod is mounted in an open guide groove of the shaft and the push-pull rod can be folded out of this guide groove of the shaft for purposes of cleaning. This design of the medical instrument definitely facilitates cleaning and the assembly and disassembly of the instrument. In addition the construction is clearly simpler and thus also more economical to produce than is possible with known tubular shaft instruments.

The disadvantage of this known construction, however, is that at least the coupling of the push-pull rod with the rotatable jaw member of the tool causes a vertical mounting of the push-pull rod perpendicular to the direction of motion of the push-pull rod when the push-pull rod rotates the jaw member between the closed and open positions. Because of this vertical mounting, in which the push-pull rod extends outside the guide groove of the shaft, a medical instrument of this type is not suited for use in endoscopic surgery, because it can result in jamming inside the trocar sleeve.

Consequently it is the aim of the invention to design a medical cutting and/or holding instrument of the aforementioned type in such a way that it can be used as well for endoscopic purposes.

### SUMMARY OF THE INVENTION

The fulfillment of this aim according to the invention is characterized in that the push-pull rod, both on the proximal and distal ends, is coupled with free play with the rotatable components in a direction essentially perpendicular to the direction of motion of the push-pull rod.

Because of the invention's coupling of the push-pull rod with play on both ends, it is possible that the rotation of the jaw member causes exclusively an axial sliding of the push-pull rod inside the guide channel of the shaft by means of an actuation of the rotatable gripping member of the handle. The play in coupling in a direction essentially perpendicular to the direction of motion of the push-pull rod makes it possible for the coupling points of the connected components running in each case on an arched course to follow just these arched tracks, but without at the same time causing a vertical mounting of the push-pull rod. A medical cutting and/or holding instrument of this design can also be inserted without problems into a narrow trocar sleeve and is thus suited for use in endoscopic surgery.

According to a practical embodiment of the invention it is proposed that the mounting point on each of the components that are to be coupled to one another is configured as a longitudinal hole to provide the coupling free play at both ends.

To simplify the coupling of the push-pull rod with the rotatable gripping member, it is further proposed with the invention that the distal-end longitudinal hole is configured as an open locking groove, which allows the push-pull rod to be hooked onto and unhooked again from the coupling with the gripping member in simple manner.

According to a first embodiment of the invention for configuring the coupling mechanism for dissolubly connecting the push-pull rod with the rotatable gripping member, it is proposed that this coupling mechanism is configured as a screw-in connection. The configuration of a screw-in connection constitutes a method of coupling between two components that is especially simple to produce and to operate.

For configuring the screw-in connection, the stem end of the rotatable gripping member is preferably configured in a forked shape for inserting the push-pull rod, and a threaded socket for inserting a threaded stud is configured in the push-pull rod or in the stem end of the rotatable gripping member and in the respective other component the longitudinal hole is configured for inserting and guiding the threaded stud.

According to a second embodiment of the invention for configuring the mechanism for dissolubly connecting the push-pull rod with the rotatable gripping member, it is proposed that the coupling mechanism for dissolubly connecting the push-pull rod with the rotatable gripping member takes the form of a pin-slot control, which includes a coupling rod positioned on the push-pull rod or on the stem end of the rotatable gripping member and an open catch groove configured in the other respective component for inserting and guiding the coupling rod. Because of the configuration of the distal-end longitudinal hole as an open catching groove, the push-pull rod can be folded easily and quickly out of the guide groove of the shaft for cleaning purposes.

To secure the push-pull rod in the working position coupled with the rotatable gripping member and to avoid accidental dissolution of the coupling, the invention proposes that the push-pull rod can be fixed in the working position coupled with the rotatable gripping member by means of a catching element, so that the catching element is preferably, in addition, pre-tensioned by a spring element in the direction of the catching position.

In addition to the use in medical cutting and/or holding instruments with a rotatable gripping member of the handle, the invention also relates to generic medical cutting and/or holding instruments with a gripping member that can slide axially in the shaft direction. In order to make a medical instrument equipped with an axially slidable gripping member also usable in endoscopic surgery, it is proposed with the invention that the push-pull rod on the distal end is coupled with the rotatable jaw member with free play in a direction essentially perpendicular to the direction of motion of the push-pull rod.

With this embodiment it is sufficient to provide only the distal-end coupling with free play, because the gripping member is mounted on the instrument shaft exclusively so that it can slide axially.

According to a practical embodiment of the invention it is further proposed with the invention that, with this type of configuration, the coupling mechanism for dissoluble connection of the push-pull rod with the slidable gripping member is configured as a driver rod that is directed perpendicularly to the direction of motion of the push-pull rod and supported on the other end on the slidable gripping member.

According to a preferred embodiment of the invention, a longitudinal hole for inserting and guiding the driver rod is configured on the shaft and the driver rod is preferable configured as a rod that can rotate and is equipped with a bolt plate.

It is finally proposed with the invention that the slidable gripping member is pre-tensioned in the position to open the tool by a spring element that engages on the driver rod, in order to facilitate opening of the tool contrary to the closing of the tool caused by the pressing together of the gripping members.

Additional characteristics and advantages of the invention can be seen from the appended illustrations, in which four embodiments of a medical cutting and/or holding instrument according to the invention are presented merely in schematic form.

### BRIEF DESCRIPTION OF THE DRAWINGS

FIG. 1 shows a partly cut-out side view of a first embodiment of a medical cutting and/or holding instrument according to the invention.

FIG. 2a shows a side view of a second embodiment of a medical cutting and/or holding instrument according to the invention.

FIG. 2b shows en enlarged, partly cut-out detail view of the medical instrument of FIG. 2a.

FIG. 3 shows a partial section along the line III-III of FIG. 2b.

FIG. 4a shows a cut-out side view of a third embodiment of a medical cutting and/or holding instrument according to the invention.

FIG. 4b shows an enlarged detail view of the detail IVb of FIG. 4a.

FIG. 5 shows a cut-out side view of a fourth embodiment of a medical cutting and/or holding instrument according to the invention.

### DETAILED DESCRIPTION OF THE INVENTION

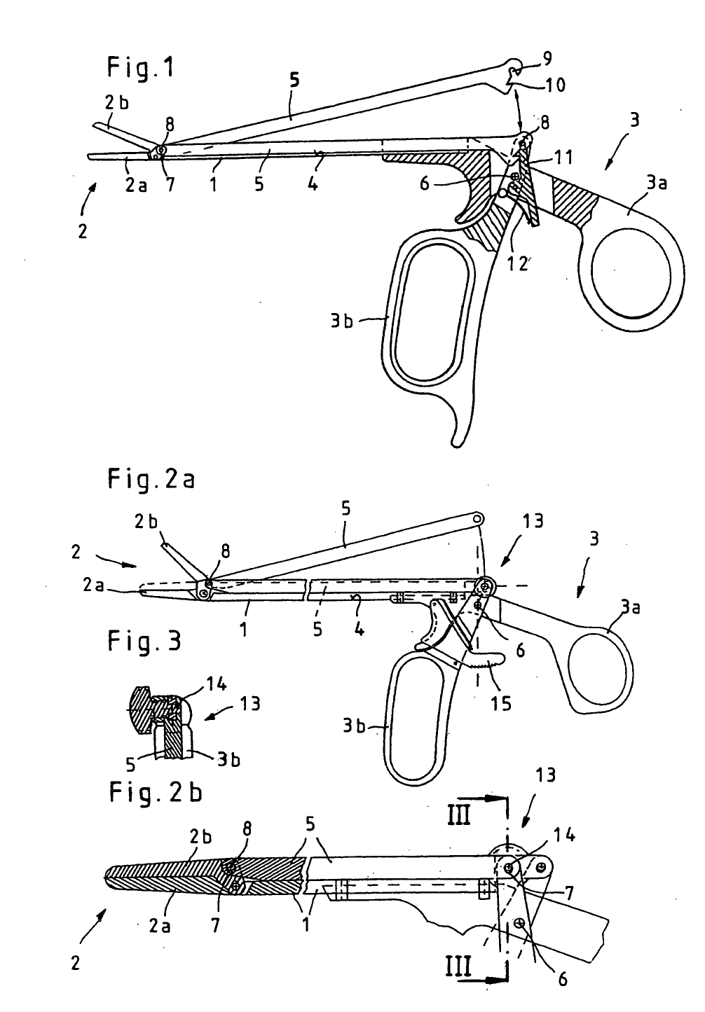

The medical cutting and/or holding instruments illustrated in FIGS. 1, 2a, 4a, and 5 consist essentially of a stem-like shaft, a tool 2 positioned on the distal end of the shaft 1 and consisting of two jaw members 2a and 2b, a handle 3 mounted on the proximal end of the shaft 1 and equipped with two gripping members 3a and 3b, as well as a push-pull rod 5 that is mounted in form-fitting manner so that it can slide axially in a guide groove 4 of the shaft 1 to actuate the tool 2 by means of the handle 3.

As can further be seen from the illustrations, the tool 2 consists of a rigid jaw member 2a, firmly connected with the shaft 1, and a jaw member 2b that can rotate by means of the push-pull rod 5. The handle 3 also has a rigid gripping member 3a that is firmly connected with the shaft 1 and a gripping member 3b that can move with respect to the rigid gripping member 3a. The rotatable jaw member 2b and the movable gripping member 3b are coupled to one another by means of the push-pull rod 5, so that the push-pull rod 5 and the rotatable gripping member 3b can be connected to one another by a coupling mechanism in such a way that the push-pull rod 5, for instance for purposes of cleaning, can be swung upward out of the guide groove 4 of the shaft 1.

With the medical instruments shown in FIGS. 1 to 4b, while the movable jaw member 3b can rotate around a pivot axis 6 with respect to the rigid gripping member 3a, FIG. 5 shows an embodiment in which the movable gripping member 3b is configured as axially slidable in the shaft direction.

To prevent the push-pull rod 5 from being raised upward out of the guide groove 4 of the shaft 1 by the rotatable gripping member 3b of the handle 3 upon actuation of the tool 2, because the mounting points both of the rotatable jaw member 2b and of the rotatable gripping member 3b, in their positioning between the two end positions, describe an arched path, the two-sided mounting points of the push-pull rod 5 in the embodiments illustrated in FIGS. 1 to 4b are configured with coupling free play.

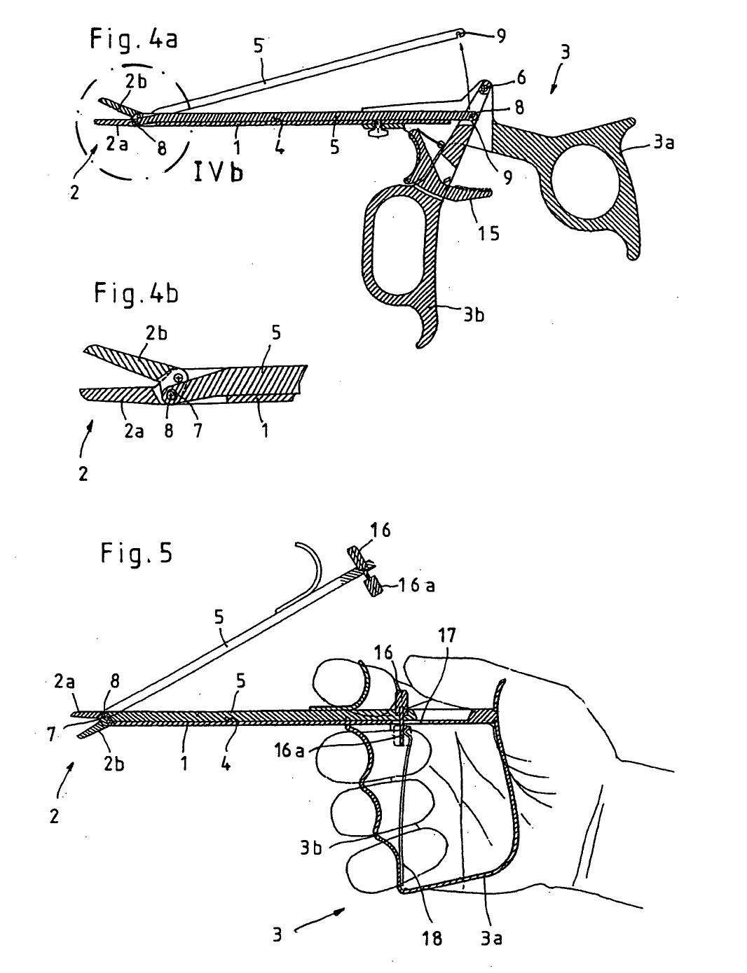

This free play in coupling in this embodiment results from the fact that the double-sided mounting points of the push-pull rod 5 are configured as longitudinal holes 7, and engage in the corresponding coupling rod 8 of the rotatable jaw member 2a and of the rotatable gripping member 3b. As can further be seen from FIGS. 1 and 4a, it is also possible to configure the proximal-end longitudinal hole 7 as an open catch groove 9, which allows an especially simple and rapid coupling of the push-pull rod 5 and the rotatable gripping member 3b.

The free play in the coupling rods 8 in the longitudinal holes 7 makes it possible for the actuation of the rotatable gripping member 3b to cause an exclusively axial sliding of the push-pull rod 5, so that a medical cutting and/or holding instrument of this configuration can also be actuated free of jamming in a trocar sleeve, and thus this instrument can also be used in endoscopic surgery.

As an alternative to the illustrated arrangement of the longitudinal holes 7 on the push-pull rod 5 and of the coupling rods 8 on the rotatable components 2b and 3b, it is of course also possible to configure the longitudinal holes 7 on the rotatable components 2b and 3b and the coupling rods 8 on the push-pull rod 5.

It is precisely in using these medical cutting an/or holding instruments for endoscopic surgery that it is advantageous if the shaft 1 with the form-fitting push-pull rod 5 inserted in the guide groove 4 has a round cross-section, because this cross-sectional shape facilitates insulation of the shaft 1 in a trocar sleeve, for instance through a ring insulation that surrounds the shaft 1, to maintain insufflation pressure. It is also possible, of course, to have all other shapes of cross-section in which the push-pull rod 5 inserted into the guide groove 4 forms a form-fitting outer contour to the shaft 1.

In addition to the different ways of mounting the movable gripping member 3b of the handle 3, that is as rotatable gripping member 3b in FIGS. 1 to 4b or as axially slidable gripping member 3b in FIG. 5, the embodiments of medical cutting and/or holding instruments shown in FIGS. 1 to 5 also differ in the way the coupling mechanism is configured for dissolubly connecting the push-pull rod 5 with the movable gripping member 3b.

In the first embodiment, shown in FIG. 1, the coupling mechanism for dissolubly connecting the push-pull rod 5 with the rotatable gripping member 3b is configured as a pin-slot control, which includes a coupling rod 8 mounted on the stem end of the rotatable gripping member 3b and an open catch groove 9 configured on the proximal end of the push-pull rod 4 for inserting and guiding the coupling rod 8.

The illustration shows the push-pull rod 5 first in the assembly and cleaning position rotated upward out of the guide groove 4 of the shaft 1 and then in the working position coupled with the rotatable gripping member 3b.

To secure the push-pull rod 5 in the coupled working position and to prevent accidental uncoupling, the push-pull rod 5 in the illustrated embodiment can be fixed in the coupled position by means of a catching element 11 that engages in a catch slot, so that the catching element 11 is pre-tensioned by a spring element 12 in the direction of the catching position.

To undo the coupling of the push-pull rod 5 and the gripping member 3b, it is necessary first to rotate the catching element 11 against the force of the spring element 12 out of its engagement with the catch slot 10. Then the push-pull rod 5 can be unfolded upward out of the guide groove 4 of the shaft 1.

In the second embodiment, shown in FIGS. 2a to 3, the coupling mechanism for dissolubly connecting the push-pull rod 5 with the rotatable gripping member 3b is configured as a screw-in connection 13. For the configuration of the screw-in connection 13, the stem end of the rotatable gripping member 3b is configured in forked shape for insertion of the push-pull rod 5 and a threaded socket is configured in the push-pull rod 5 for inserting a threaded stud 14 of the screw-in connection 13. In the two fork-shaped ends of the rotatable gripping member 3b, corresponding longitudinal holes 7 are provided for inserting and guiding the threaded stud 14, as can be seen in particular from FIGS. 2b and 3.

As also to be seen from FIG. 2a, the rotatable gripping member 3b of the handle 3 can be fixed by a preferably spring-loaded stopping device 15 in the rotated position with respect to the rigid gripping member 3a.

In the third embodiment, shown in FIGS. 4a and 4b, the coupling mechanism for dissolubly connecting the push-pull rod 5 with the rotatable gripping member 3b is again configured as a pin-slot control. Contrary to the first embodiment, shown in FIG. 1, the push-pull rod 5 in this configuration is not fixed in the coupled position by a catching element 11. In addition, the two embodiments differ from one another in the manner of mounting the two gripping members 3a and 3b to one another as well as in the position of the mounting points of the push-pull rod 5 on the rotatable jaw member 2b.

The fourth embodiment, illustrated in FIG. 5, finally, shows a medical cutting and/or holding instrument with an axially slidable gripping member 3b. Because in this configuration the proximal-end mounting point of the push-pull rod 5 is non-problematic because of the axial slidability of the gripping member 3b, it is sufficient that the push-pull rod 5 on the distal end is coupled with the rotatable jaw member 2b with free play in a direction essentially perpendicular to the direction of motion of the push-pull rod 5, in order to prevent the push-pull rod 5 from being raised out of the guide groove 4 of the shaft 1.

In this configuration the coupling mechanism for dissolubly connecting the push-pull rod 5 with the slidable gripping member 3b is configured as a drive rod 16 directed essentially perpendicularly to the direction of motion of the push-pull rod 5, and this drive rod 16 is mounted on the push-pull rod 5 on one end and supports itself on the other end on the slidable gripping member 3b.

As can be seen from the illustration, in the shaft 1 a longitudinal hole 17 is formed for inserting and guiding the drive rod 16. To secure the push-pull rod 5 in the coupled position, the drive rod 16 has a bolt plate 16a, which after insertion into the longitudinal hole 17 of the shaft 1 can be rotated 90 degrees and thus blocks any raising of the push-pull rod 5. In the illustrated embodiment the bolt plate 16a also serves as a stopping face for a spring element 18, by means of which the slidable gripping member 3b is pre-tensioned in the position that opens the tool 2.

The medical cutting and/or holding instruments configured as shown in the illustrations are distinguished in that the push-pull rod 5 that is form-fitted in the guide groove 4 of the shaft 1, first, can be easily and quickly folded out of the guide groove 4 for purposes of assembly and cleaning and, second, the positioning of the push-pull rod 5 by means of the movable gripping members 3b of the handle 3 causes the push-pull rod 5 to be mounted running exclusively in the axial direction of the shaft 1 because of the mounting of the push-pull rod 5 with free play. For this reason, medical instruments of this configuration can be actuated also without clamping in narrow trocar sleeves, as used in endoscopic surgery.

1. A medical cutting and/or holding instrument having a shaft, a handle consisting of two gripping members that is mounted on the proximal end of the shaft, and a push-pull rod, mounted in a guide groove of the shaft, to actuate a tool consisting of two jaw members positioned on the distal end of the shaft, such that for opening and closing the tool the push-pull rod can be coupled on the distal end with at least one rotatable jaw member of the tool and on the proximal end with a rotatable gripping member of the handle, and such that the push-pull rod and the rotatable gripping member can be dissolubly connected with one another by means of a coupling mechanism, characterized in that the push-pull rod both on the proximal and on the distal ends is coupled with free play with the rotatable components essentially perpendicular to the direction of motion of the push-pull rod.

2. A medical cutting and/or holding instrument according to claim 1, characterized in that to ensure the two-sided coupling free play, the support point is configured as a longitudinal hole on one of the respective components that are to be coupled with one another.

3. A medical cutting and/or holding instrument according to claim 2, characterized in that the distal-end longitudinal hole configured as an open catch groove.

4. A medical cutting and/or holding instrument according to any claim 1, characterized in that the coupling mechanism for dissolubly connecting the push-pull rod with the rotatable gripping member is configured as a screw-in connection.

5. A medical cutting and/or holding instrument according to claim 4, characterized in that the stem end of the rotatable gripping member is configured in a fork shape for inserting the push-pull rod.

6. A medical cutting and/or holding instrument according to claim 4, characterized in that a threaded socket for inserting a threaded stud is configured in the push-pull rod or in the stem end of the rotatable gripping member and in the other respective component the longitudinal hole is configured for inserting and guiding the threaded stud.

7. A medical cutting and/or holding instrument according to claim 1, characterized in that the coupling mechanism for dissolubly connecting the push-pull rod with the rotatable gripping member takes the form of a pin-slot control, which includes a coupling rod positioned on the push-pull rod or on the stem end of the rotatable gripping member and an open catch groove configured in the other respective component for inserting and guiding the coupling rod 8.

8. A medical cutting and/or holding instrument according to claim 1, characterized in that the push-pull rod can be fixed by means of a catching element in the working position coupled with the rotatable gripping member.

9. A medical cutting and/or holding instrument according to claim 8, characterized in that the catching element is pre-tensioned by a spring element in the direction of the catching position.

10. A medical cutting and/or holding instrument having a shaft, a handle consisting of two gripping members positioned on the proximal end of the shaft, and a push-pull rod mounted in a guide groove of the shaft for actuating a tool that is positioned on the distal end of the shaft and that consists of two jaw members, such that for opening and closing the tool the push-pull rod can be coupled on the distal end with at least one rotatable jaw member of the tool and on the proximal end with a gripping member of the handle that can slide axially in the shaft direction, and such that the push-pull rod and the axially slidable gripping member can be dissolubly connected to one another by a coupling mechanism, characterized in that the push-pull rod is coupled on the distal end with free play with the rotatable jaw member in a direction essentially perpendicular to the direction of motion of the push-pull rod.

11. A medical cutting and/or holding instrument according to claim 10, characterized in that the coupling mechanism for dissolubly connecting the push-pull rod with the slidable gripping member is configured as a drive rod that is directed essentially perpendicularly to the direction of motion of the push-pull rod and that, first, is mounted on the push-pull rod and, second, supports itself on the slidable gripping member.

12. A medical cutting and/or holding instrument according to claim 11, characterized in that a longitudinal shaft is configured in the shaft for inserting and guiding the drive rod.

13. A medical cutting and/or holding instrument according to claim 12, characterized in that the drive rod is configured as a rotatable rod equipped with a bolt plate.

14. A medical cutting and/or holding instrument according to claim 11, characterized in that the slidable gripping member is pre-tensioned in the position that opens the tool by a spring element engaging on the drive rod.
